(19) United States
(12) Patent Application Publication
(10) Pub. No.: US 2026/0157652 A1
(43) Pub. Date: Jun. 11, 2026

(54) Method for Monitoring a Volumetric Flow of Respiratory Air in an Analyzer

(71) Applicant: Robert Bosch GmbH, Stuttgart (DE)

(72) Inventors: Christoph Beck, Esslingen (DE); Klaus Mueller, Stuttgart (DE)

(21) Appl. No.: 19/150,143

(22) PCT Filed: Jan. 17, 2024
(86) PCT No.: PCT/EP2024/050949

§ 371 Date: Jul. 22, 2025

(30) Foreign Application Priority Data
Jan. 25, 2023 (DE) 10 2023 200 556.5

(51) Int. Cl. A61B 5/08; A61B 5/00; A61B 5/087; A61B 5/091; A61B 5/097

(52) U.S. Cl. CPC A61B 5/082; A61B 5/087; A61B 5/091; A61B 5/097; A61B 5/7221; A61B 5/746

(57) ABSTRACT

A method is for monitoring a volumetric flow of breathing air in an analysis device, in particular for measuring fractionated exhaled nitrogen monoxide. The breathing air is exhaled in the form of the volumetric flow preferably via an opening into the analysis device. The volumetric flow is monitored during a monitoring period for a deviation with respect to a predetermined level. The predetermined level is time-dependent.

### PRIOR ART

The level of fractional nitric oxide (NO) in exhaled air (FeNO) has become established as a measure of lung inflammation, particularly in connection with asthma. The proportion of NO in exhaled air depends significantly on the flow of air during exhalation. To measure FeNO using an analyzer, it is therefore necessary to provide the breath sample at a defined flow rate (volume flow). With the help of appropriate feedback (visual and/or acoustic) in real time, the user can control and adjust the strength of their exhalation flow. If the required flow rate cannot be maintained, sampling should preferably be interrupted by the analyzer to prevent incorrect measured values from being generated and output. One such FeNO analyzer is based on the device described in EP 1384069 B 1 and is marketed under the brand name Vivatmo®.

Usually, a constant value of +/−10% relative to a fixed volume flow of 50 milliliters per second (ml/s) is determined, as specified in the guideline for measuring FeNO (ATS/ERS recommendations for standardized procedures for the online and offline measurement of exhaled lower respiratory nitric oxide and nasal nitric oxide, 2005. Am J Respir Crit Care Med. 2005; 171(8): 912-930. doi: 10.1164/rccm.200406-710ST). Due to this rather narrow corridor, inexperienced users often need several attempts to provide a correct breath sample.

### DISCLOSURE OF THE INVENTION

### Advantages of the Invention

Against this background, the invention relates to a method for monitoring a volume flow of breathing air in an analysis device, wherein the breathing air is preferably exhaled into the analysis device in the form of the volume flow via an opening. The volume flow is monitored during a monitoring period for any deviation from a specified, time-dependent level. In particular, the method can be used to measure fractional exhaled nitric oxide (FeNO).

Furthermore, the invention relates to an analysis device set up in this way, in particular an analysis device with a correspondingly programmed control unit and a computer program, i.e., software comprising instructions which, when the program is executed by a computer, in particular by the control unit of the analysis device, causing the latter to carry out the method according to the invention.

The analysis device according to the invention and the associated method utilize the fact that deviations of the volume flow from setpoints of different magnitudes can be tolerated in different time intervals. Depending on the variable of interest to be measured in the breathing air, i.e., in particular the analyte to be measured, such as NO, CO2, or alcohol, and its physiological background, the time dependence of the level can be determined.

Preferably, the level can represent a deviation tolerance for a tolerable deviation of the volume flow from one or more setpoints. The setpoints can be, in particular, a specified volume flow, i.e., a volume moved through an area per unit of time. In the case of FeNO, for example, the above guideline specifies a volume flow of 50 ml/s.

Preferably, the level exhibits a higher deviation tolerance during a second interval of the monitoring period than during a preceding first interval. In the case of FeNO measurement, it is possible to take advantage of the fact that the part of the exhaled air relevant for the measurement, i.e., the relevant breath sample, comes from the lower airways, in particular from the bronchi, and that the parts exhaled first, namely the breathing air from the mouth and throat and from the trachea (also referred to as dead space volume), should not be taken into account for the measurement. This dead space volume causes the relevant breath sample to arrive at the sensor in the analyzer with a delay. This means that fluctuations in the flow during exhalation only have a delayed effect on the measurement signal, allowing a higher deviation tolerance to be set for the second interval, which is advantageous. This has the advantage of simplifying compliance with the correct flow or volume flow for the user. Due to the dead space volume described above, users must maintain a reasonably constant flow rate during exhalation for a relatively long time, which is particularly difficult for users with smaller lung volumes. By adjusting the tolerated level of the deviation from the specified volume flow, the strict requirements for a breath sample to be classified as correct can be relaxed. In addition, the method makes it easier to carry out several valid measurement tests in a short period of time if necessary. In addition to FeNO measurement, the method is therefore also particularly well suited for measuring other analytes in breathing air from the lower respiratory tract.

In accordance with a special design, the breathing air is exhaled into the analyzer during a bypass period and a subsequent measurement period. A measurement period is defined as a period during which the breathing air is measured by one or more sensors of the analysis device and the data recorded during this period is considered valid. A bypass period, on the other hand, is a period during which no measurements are taken or recorded measurement data is considered invalid. Depending on the specific design of the analysis device, the breathing air may be passed by the sensor without coming into contact with it during the bypass period and/or the sensor may be deactivated. In particular in the case of FeNO measurement, the monitoring period may cover at least parts of the bypass period and the measurement period. The monitoring period usually covers at least the measurement period. In the case of FeNO measurement, due to the time delay described above, part of the bypass period immediately preceding the measurement period is also covered. Depending on the specific configuration, particularly for FeNO measurement, the monitoring period may end before the end of the measurement period, so that the monitoring period covers part of the bypass period and an immediately adjacent first part of the measurement period, but does not cover a second part of the measurement period.

In an advantageous embodiment, the level increases or decreases during at least one interval of the monitoring period. The level may increase or decrease continuously, at least in sections, in particular linearly or exponentially, or in steps. For example, the level may decrease at the beginning of the first interval and increase toward the end of the second interval.

According to an advantageous further development of the invention, the analysis device issues a warning if a deviation in the volume flow during the monitoring period exceeds a predetermined value of the level. The specified value can correspond in particular to the value of the deviation tolerance. Preferably, the user is informed of the current value of the volume flow and warned of any deviation approaching or exceeding the level, for example by means of a visual and/or acoustic signal from the analysis device, in order to reduce the deviation.

### BRIEF DESCRIPTION OF THE DRAWINGS

Exemplary embodiments of the invention are shown schematically in the drawings and explained in more detail in the description hereinafter. Like reference signs are used for elements illustrated in the various drawings having a similar effect, wherein a repeated description of the elements has been omitted.

Shown are

FIG. 1 shows an exemplary embodiments of the analysis device according to the invention for analyzing breathing air,

FIG. 2 shows a breakdown of the exhalation process into a bypass period and a measurement period with a partially simultaneous monitoring period, and

FIG. 3 shows an example volume flow curve and the time-dependent level tolerance according to exemplary embodiments of the method according to the invention.

### EMBODIMENTS OF THE INVENTION

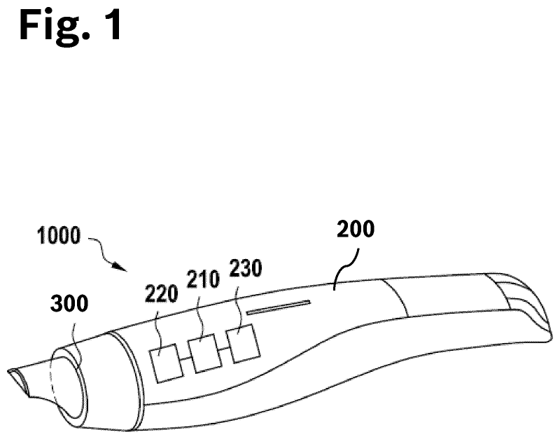

FIG. 1 shows an example embodiment of the analytical device 1000 according to the invention for analyzing breathing air, in particular for measuring fractionated exhaled nitrogen monoxide, which may be based on a device as described in patent specification EP 1384069 B1. The analysis device 1000 comprises a mouthpiece 300 through which a user can breathing air into the analysis device 1000. The mouthpiece 300 can be attached to the actual analyzer, also referred to as handheld device 200, preferably in an interchangeable manner. In addition to a suitable sensor, such as a sensor described in EP 1384069 B1 for measuring NO converted to NO₂ in the analysis device, the handheld device 200 comprises, among other things, a control unit 210 for performing the measurement and a memory in which instructions for performing the method according to the invention are stored as software. Furthermore, the handheld device 200 comprises a sensor 220 connected to the control unit 210 for measuring the correct volume flow of the exhaled air, for example the combination sensor BME280 from Robert Bosch GmbH, and preferably an output device 230 for outputting optical and/or acoustic feedback, in particular via a display or a loudspeaker, to the user.

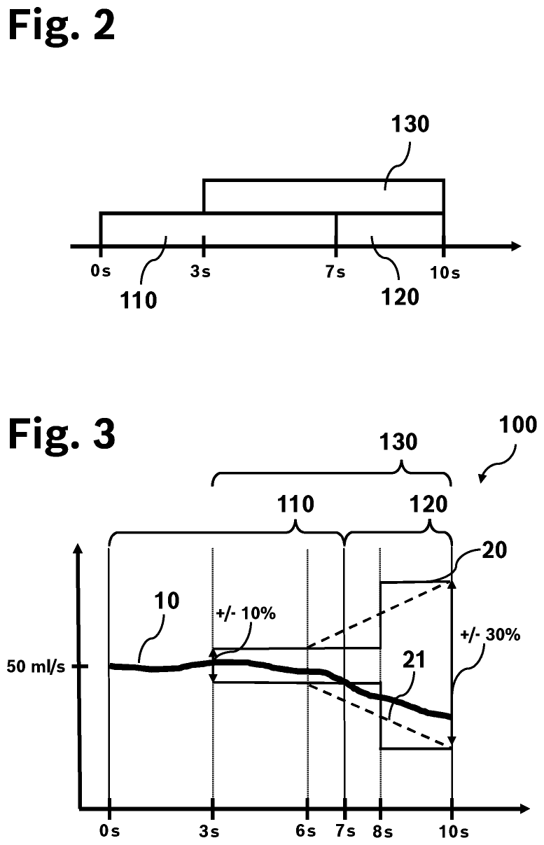

The method according to the invention 100 will now be described by way of example with reference to FIG. 2 and, in particular, FIG. 3. FIG. 2 schematically shows the time intervals that elapse during a measurement of breathing air, for example during a measurement of fractional exhaled nitrogen monoxide (FeNO) in exhaled air using a breath analyzer based on a device as described in patent specification EP 1384069 B1. The user breathes continuously into a designated opening of the analysis device 1000 for several seconds, for example 10 seconds.

The first part of the exhaled air, which is exhaled during a predefined period known as bypass period 110, should cover the part of the exhaled air originating from the dead space volume described above. The second part of the exhaled air, which follows immediately and without interruption, should consist exclusively of the breath sample relevant for the measurement, originating from the bronchi. This second part is exhaled during the predefined measurement period 120 and used for the measurement. In the following, bypass period 110 and measurement period 120 last seven seconds and three seconds, respectively, as an example.

For a breath sample to be suitable for measurement, the user must exhale as evenly as possible, especially during the measurement period. According to the above guideline, the user should provide a volume flow of 50 milliliters per second with a tolerated deviation of +/−10% for a FeNO measurement. To ensure this, the appropriately configured analysis device monitors whether the volume flow exceeds the specified tolerance level of 10% relative to 50 ml/s during a monitoring period of 130. As shown in FIG. 2, the monitoring period 130 covers the entire measurement period and a part of the bypass period preceding the measurement period. For example, the monitoring period can begin after the first three seconds of the bypass period, thus covering the remaining four seconds of the bypass period and the three seconds of the measurement period, and then end at the same time as the measurement period. Preferably, the user is informed of the current value of the volume flow and warned of any deviation approaching or exceeding the level, for example by means of a visual and/or acoustic signal from the analysis device 1000, in order to reduce the deviation. The level can also include two different values, for example a tolerance value of +/−10% and a threshold value of +/−20%, so that, for example, if the tolerance value is exceeded, only a warning is issued initially and the measurement is only classified as invalid when the threshold value is exceeded, wherein in special designs, a minimum duration for exceeding one or both values may also be specified (if necessary) for issuing the warning or classifying the measurement as invalid. The output can be acoustic and/or visual, for example as a color output, such as orange and red when the tolerance value or threshold value is exceeded, via (LED) lamps or the display.

According to the invention, the level of tolerated deviation is to be specified as a function of time. In particular, the level can be relaxed, i.e., widened, in the last seconds of the measurement period 120, as shown in FIG. 3. This is not particularly critical in the case of FeNO measurement or, in general, measurement of a breath sample from the lower respiratory tract (bronchi) due to the time delay described above, but it does make the process noticeably easier for the user. For example, the tolerance of +/−10% in the last two seconds of the measurement period 120 can be increased to +/−30%. The tolerance can be increased abruptly or gradually. FIG. 3 shows, alongside an example volume flow 10, such a time-dependent level 20, which increases abruptly from +/-10% to +/-30% eight seconds after the start of the exhalation process, i.e., one second after the start of the measurement phase. The level can also be increased in several stages, for example to 15% after the first half second, to 25% after 1.5 seconds, and to 35% after 2.5 seconds after the start of the measurement period. A steady increase can have a linear or exponential slope and can also end at a level of +/−30% or +/−35%, for example. FIG. 3 (dashed line) also shows a steady increase in level 21 to +/−30% starting six seconds after the beginning of the exhalation process, i.e., still in bypass period 110 and one second before the start of measurement period 120. In the example shown in FIG. 3, the volume flow 10 of the exhaling user would deviate more strongly than the specified level between seconds 7 and 8 despite the sudden relaxing 20 from second 8, whereas in the alternative example with continuous relaxing 21 from second 6, it would remain within the tolerance range. If the level comprises several values as described above, these values can preferably be increased in parallel, to the same or alternatively to different extents. If, in particular, an associated measurement uncertainty is acceptable, the increase in the level can also begin during the last part of the bypass period, as shown in FIG. 3 for the steady increase 21 from second 6, and, for example, an increase in smaller steps or with a lower gradient can occur. Alternatively or additionally, the monitoring period 130 may also end before the end of the measurement period 120, for example half a second or a whole second before the end of the measurement period, which would be equivalent to an infinitely large level.

Thus, as illustrated in these examples, the deviation tolerance level can be increased during a second interval of the monitoring period (time interval between seconds 6 or 8 and 10 in the examples shown in FIG. 2), wherein the second interval can begin during the measurement period or already during the bypass period, depending on the application and the load capacity of the measurement. During the first interval of the monitoring period, the level may remain constant (time interval between seconds 1 and 8 or 6 in the examples shown in FIG. 2).

Alternatively, the level may also change during the first interval. For example, the level could be set higher at the beginning of the first interval and thus at the beginning of the monitoring period, for example at +/−20% or +/−15%, and then reduced, either in steps or continuously, to +/−10% if an increased tolerance at the beginning of the exhalation process is acceptable for the measurement.

1. A method for monitoring a volumetric flow of breathing air in an analysis device for measuring fractionated exhaled nitrogen monoxide, the method comprising:
exhaling the breathing air as the volumetric flow through an opening into the analysis device; and
monitoring the volumetric flow during a monitoring period for a deviation with respect to a time-dependent predetermined level.

2. The method according to claim 1, further comprising:
exhaling the breathing air into the analysis device during a bypass period and a subsequent measurement period; and
changing the time-dependent predetermined level during the subsequent measurement period

3. The method according to claim 1, wherein:
the time-dependent predetermined level represents a deviation tolerance for a tolerable deviation of the volumetric flow from one or more setpoints,
the time-dependent predetermined level has a higher deviation tolerance during a second interval of the monitoring period than during a temporally preceding first interval, and
the second interval lies in the a measurement period.

4. The method according to claim 1, wherein:
the monitoring period covers a part of a bypass period, and the part immediately precedes a measurement period

5. The method according to claim 1, wherein the monitoring period covers a part of a measurement period.

6. The method according to claim 1, wherein the monitoring period ends before an end of a measurement period.

7. The method according to claim 1, wherein the time-dependent predetermined level increases continuously at least during an interval of the monitoring period.

8. The method according to claim 7, wherein the increase in the time-dependent predetermined level is linear or exponential at least in sections.

9. The method according to one claim 7, wherein the increase in the time-dependent predetermined level is stepped at least in sections.

10. The method according to claim 1, wherein the time-dependent predetermined level decreases continuously at least during an interval of the monitoring period.

11. The method according to claim 1, further comprising:
issuing a warning using the analysis device when the deviation of the volumetric flow during the monitoring period exceeds a predetermined value of the time-dependent predetermined level.

12. An analyzer for analyzing breathing air for measuring fractionated exhaled nitric oxide, wherein the analyzer is configured to carry out the method according to claim 1.

13. The method according to claim 1, wherein a computer program comprises instructions which, when executed by a computer, cause the computer to execute the method.
